(19) United States
(12) Patent Application Publication
(10) Pub. No.: US 2022/0175673 A1
(43) Pub. Date: Jun. 9, 2022

(54) SURGICAL DEVICE

(71) Applicant: Saint Anthony Biomedical, LLC, Chattanooga, TN (US)

(72) Inventors: Mathew T. Epps, Chattanooga, TN (US); Mark A. Brzezienski, Chattanooga, TN (US)

(21) Appl. No.: 17/410,692

(22) Filed: Aug. 24, 2021

Related U.S. Application Data
(63) Continuation of application No. 16/168,940, filed on Oct. 24, 2018, now abandoned.
(60) Provisional application No. 62/576,354, filed on Oct. 24, 2017.

(51) Int. Cl. A61K 9/08; A61K 45/06; A61P 31/04; A61M 3/02; A61L 26/00; A61L 24/00; A61K 31/155; A61K 9/00; A61L 31/16

(52) U.S. Cl. CPC A61K 9/08; A61K 45/06; A61P 31/04; A61M 3/0204; A61L 26/0028; A61K 47/18; A61L 24/0015; A61K 31/155; A61K 9/0014; A61L 31/16; A61L 26/0066; A61L 26/0004

(57) ABSTRACT

An improved surgical device is disclosed herein. The improved surgical device includes a delivery device and a sealed container. The sealed contained is prepositioned internal to the delivery device and includes an antimicrobial solution. The improved surgical device is configured for inserting an implant into a surgical site. In some embodiments, the sealed container is configured to be manually broken to release the antimicrobial solution prior to inserting the implant into the delivery device. In other embodiments, the sealed container is configured to be automatically broken to release the antimicrobial solution prior to inserting the implant into the delivery device.

### CROSS REFERENCE TO RELATED APPLICATIONS

This application claims the benefit of priority to U.S. provisional application No. 62/576,354, filed Oct. 24, 2017, which is incorporated herein by reference in its entirety.

### BACKGROUND

Preventing infection in wounds and surgical cites is of utmost importance. Infections can cause serious complications, delay healing, impair cosmetic outcomes, and increase healthcare costs. In addition to implant-related surgical site infection, another issue, particularly arising with the use of implants, is the formation of excess scar tissue around the implant. Scar tissue can harden and lead to tightening around or squeezing of the implant, a phenomenon known as capsular contracture. Capsular contracture can lead to an implant that is misshapen, painful, and hard and can result in an unnatural appearance and feel. While the mechanism is still under investigation, capsular contracture appears to be more common following infection, which can occur anytime from several days to several years after surgery or implantation. In particular, subclinical infection is thought to be a main contributor to capsular contracture. Clinical or frank infection is a major contributor to implant explanation. Infection occurs when bacteria colonize a surface (implant) with or without biofilm formation. Subclinical infection does not produce the signs and symptoms traditionally associated with frank infection (such as pain, tenderness, fever, and pus) and manifests itself as a chronic inflammatory response. This inflammatory response leads to an overaggressive collagen deposition during tissue remodeling resulting in fibrous tissue buildup and capsule rigidity with eventual implant distortion.

Current treatments to avoid both infection and capsular contracture include antibiotic washings of the wound/surgical site as well as the implant itself at the time of surgery. Oral or intravenous antibiotics can also be administered prior to, during and after surgery. While irrigation of wounds and surgical sites with antibiotic agents would seem to be a logical step for fighting infections, it is a much debated topic.

Adams and colleagues sought to reduce both infection and capsular contracture caused by infection by irrigating the surgical site with a “triple antibiotic solution” containing a mixture of bacitracin, gentamycin, and cefazolin. Adams subsequently published results of a six-year clinical study showing that patients who received surgeries incorporating these techniques have a 1% capsular contracture rate as opposed to national rates, which approached 15-20% in that same time period (Adams et al. (2006) Plast. Reconstr. Surg. 117130-36). More recently, however, other studies have shown that irrigation with the triple antibiotic solution had only minor effects and was not associated with significant reduction in capsular contracture rate (3.7% with vs. 3.6% without) (Headon et al. (2015) Arch. Plast. Surg. 42:532-543).

Others have tried different irrigation solutions, such as Hall-Findlay, who disclosed the use of a dilute marcain/betadine (povidone iodine) solution to irrigate a breast pocket before breast implantation (Hall-Findlay (2010) PRS J. 127(1):56-66). Further examples of antibiotics that have been used alone or in combination with others for irrigation purposes are neomycin, bacitracin, polymyxin, cefazolin, kanamycin, gentamicin and vancomycin (Yalanis et al. (2015) PRS J. 136:687-698).

Twomey (Infection Control Today 2013) discloses that presentations at the 2012 annual meetings of both the American College of Surgeons and the Association for Professionals in Infection Control and Epidemiology (APIC), an FDA-cleared wound cleansing and debridement system with 0.05% chlorhexidine gluconate (CHG) for irrigation was reported. Testing of the effectiveness of 0.05 CHG against selective multidrug resistant (MDR) surgical pathogens showed greater than or equal to 99.99% log-reduction in MDR isolates (MRSA, E. faecium, K pneumoniae, E. aerogenes, E. coli and A. baumani) following 1-minute exposure to 0.05% CHG.

U.S. Pat. No. 7,959,617 similarly discloses the use of a chlorhexidine gluconate solution to irrigate a wounds for preventing infection. After irrigation, the wound is rinsed with sterile saline or water to remove the chlorhexidine gluconate from the site. Chlorhexidine has been associated with allergic contact dermatitis, tissue toxicity, and anaphylaxis (Lachapelle, (2014) Eur. J. Dermatol. 24(1):3-9). Chlorhexidine has also been reported to have a cytotoxic effect on keratinocytes and is suggested as having an inhibitory effect on wound healing (Totoraitis (2017) J. Drugs Dermatol. 16(3):209-212). Thus removal of chlorhexidine from a wound or surgical site is standard.

Given the constant pressure to fight infections and reduce complications associated with infections like capsular contracture, new methods, compositions, and articles are needed. The compositions and methods disclosed herein address these and other needs.

### SUMMARY

In accordance with the purposes of the disclosed methods, compositions, and articles, as embodied and broadly described herein, the disclosed subject matter, in one aspect, relates to compositions and methods of making and using compounds and compositions. In further aspects, the disclosed subject matter relates to articles coated with the disclosed compositions. In specific aspects, the disclosed subject matter relates to methods of preventing bacterial growth in a wound or surgical site of a subject comprising irrigating or coating the wound or surgical site with a solution comprising a biguanide derivative, and then closing or covering the wound or surgical site without washing the solution from the wound or surgical site with saline or water. Specific solutions comprising a biguanide derivative are also disclosed herein.

In further aspects, the disclosed subject matter relates to articles that are coated with a solution comprising a biguanide derivative. The articles can be, for example, an implant, an apparatus for inserting an implant through an opening into a surgical cavity, tubing, sutures, and many others. Methods of coating the disclosed articles with a solution comprising a biguanide derivative are also disclosed herein.

Additional advantages of the disclosed subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1A illustrates the lateral view of an inframammary surgical incision for prosthetic device insertion during breast augmentation mammaplasty.

FIG. 1B illustrates various typical devices, silicone and saline, and device shapes, shaped and round, used during cosmetic prosthetic augmentation of the breast.

FIG. 1C illustrates a tissue expander containing a fill port and anchoring tabs typically implanted during breast cosmetic and reconstructive procedures.

FIG. 2A illustrates a surgically placed cavity drain which utilizes a biguanide coating and also illustrates a raised portion of the drain at the point of exit as well as a variety of suture anchor grooves or channels.

FIG. 2B illustrates the transcutaneous view of surgically placed cavity drain whereby the drain possesses a raised portion at its point of exit from the body cavity to the skin.

FIG. 2C illustrates both the lateral views of an inframammary surgical incision for prosthetic device insertion during breast surgery with a breast pocket drain (left), and a surgical drain suture anchored to the skin utilizing molded grooves on the surface of the drain (right).

FIG. 3A illustrates the top view of a funnel-device, containing a breast prosthesis and an internal sterile-sealed irrigation/lubricant solution pouch, used to ease and insertion of a breast implant through a surgical incision and promote sterile technique.

FIG. 3B illustrates the side-view of a funnel-device with a breast prosthesis in position for insertion. The illustrated device contains a port for sterile introduction of irrigation/lubrication solution which facilitates the implant movement through the device at the distal end. Also illustrated is the technique whereby the irrigation/lubricant is directly poured into the funnel-device.

FIG. 4A illustrates a lateral view (left) and front view (right) of the breast with an anchor or Wise-pattern shaped occlusive dressing in the pattern of a typical breast reduction surgery.

FIG. 4B illustrates a lateral view (left) and front view (right) of the breast with an elliptical or oval shaped occlusive dressing in the pattern of a typical breast reduction surgery or breast reconstruction procedure.

FIG. 4C illustrates a front view of the breast with a circular shaped occlusive dressing overlying the nipple-areolar complex and possessing a raised tab or blister designed to facilitate griping during dressing removal.

FIG. 4D illustrates the lateral view of an oval shaped occlusive dressing intended to be placed over a surgical or traumatic wound.

### DETAILED DESCRIPTION

The compositions, methods, and articles described herein may be understood more readily by reference to the following detailed description of specific aspects of the disclosed subject matter and the Examples included therein.

Before the present compositions, methods, and articles are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the solution” includes mixtures of two or more such solutions, reference to “an agent” includes mixture of two or more such agents, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. In preferred aspects, a subject is a human.

By “reduce” or other forms of the word, such as “reducing” or “reduction,” is meant lowering of an event or characteristic (e.g., bacterial growth). It is understood that this is typically in relation to some standard or expected value, in other words it is relative, but that it is not always necessary for the standard or relative value to be referred to. For example, “reduces bacterial growth” or “reduces capsular contracture” means reducing the rate of growth of a bacteria relative to a standard or a control or reducing the incidence of capsular contracture.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., bacterial growth or capsular contracture). The term “control” is used synonymously with the term “treat.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Reference will now be made in detail to specific aspects of the disclosed materials, compounds, compositions, articles, and methods, examples of which are illustrated in the accompanying Examples.

### Compositions and Methods

In specific aspects, disclosed herein are methods of preventing bacterial growth in a wound or surgical site of a subject comprising irrigating or coating the wound or surgical site with a solution comprising a biguanide derivative, and then closing or covering the wound or surgical site without washing the solution from the wound or surgical site with saline or water. The solution can be an aqueous solution. The solution can be made from a solid (tablet, capsule, pellet), powder, or dissolvable membrane to be reconstituted with an aqueous solution prior to use. The solution can also comprise a tissue adhesive.

In certain aspects, the biguanide derivative can be a bisbiguanide derivative. In specific examples, the bisguanide derivative can be chlorhexidine. Chlorhexidine is a chemical antiseptic, and it combats both gram positive and gram negative microbes. It is bacteriostatic, hampering the growth of bacteria, and bacteriocidal, killing bacteria. It is often used as an active ingredient in mouthwash designed to kill dental plaque and other oral bacteria. It is also used for general skin cleansing, as a surgical scrub, and as a preoperative skin preparation. Chlorhexidine is typically used in the form of acetate, gluconate, or hydrochloride, either alone or in combination with other antiseptics such as cetrimide. Hibiclens (4% w/v chlorhexidine gluconate soap) is an antiseptic agent used in the pre-operative period as a body soap to decolonize MRSA carriers. Hibiclens soap is also a commonly used surgical prep. Literature in orthopedic surgery and wound cleansing reveals the use of a 0.05% chlorhexidine gluconate aqueous solution as a wound decontaminate and antiseptic agent.

Chlorhexidine is highly active against a variety of Gram-positive aerobic bacteria, including Streptococcus mutans, S. pyogenes (group A [3-hemolytic streptococci), S. salivarius, and S. sanguis. Chlorhexidine is active against Staphylococcus aureus, S. epidermidis, S. haemolyticus, S. hominis, and S. simulans. The drug is active against both oxacillin-resistant (ORSA) and oxacillin-susceptible staphylococci (also known as methicillin-resistant [MRSA] or methicillin-susceptible staphylococci). Chlorhexidine is active against Enterococcus, including E. faecalis and E. faecium, and is active against both vancomycin-susceptible and vancomycin-resistant strains. Chlorhexidine is active against some anaerobic bacteria. The drug is active against some strains of Bacteroides, Propionibacterium, Clostridium deficile, and Selenomonas, but is less active against Veillonella. Chlorhexidine has some activity against Candida albicans, C. dubliniensis, C. glabrata (formerly Torulopsis glabrata), C. gullermondii, C. pseudotropicalis, C. krusei, C. lusitaniae, and C. tropicalis. Chlorhexidine also has some activity against dermatophytes, including Epidermophyton occosum, Microsporum gypseum, M canis, and Trichophyton mentagrophytes. Chlorhexidine appears to have antiviral activity against viruses that have a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, and variola virus (smallpox virus).

Specific examples of bisbiquanide derivatives that can be used in the disclosed compositions, methods, and articles are chlorhexidine gluconate, chlorhexidine digluconate, chlorhexidine acetate, chlorhexidine diacetate, chlorhexidine dehydrate, chlorhexidine hexa-metaphosphate, chlorhexidine metaphosphate, chlorhexidine trimetaphosphate, alexidine, polyaminopropyl biguanide, polyhexamide biguanide, polyhexamethylene biguanide hydrochloride, and any mixture thereof. In some specific examples, the solution comprise chlorhexidine gluconate. On other specific examples, the solution comprises chlorhexidine hexametaphosphate. In other examples, the solution comprises chlorhexidine hexametaphosphate, which can be prepared by combining chlorhexidine gluconate with sodium hexametaphosphate.

In other aspects, the biguanide derivative can be linked to an additional molecule and/or substrate. For example, in some aspects the biguanide derivative can be intercalated in, or covalently or ionically bound to, a montmorillonite, copper (II)/montmorillonite composite, chitosan-montmorillonite composite, hexa-metaphosphate (HMP) nanoparticle/nanofiber, hexa-metaphosphate (HMP) glass ionomer, triphosphate (TP) nanoparticle/nanofiber, tri-metaphosphate (TMP) nanoparticle/nanofiber, acetate (CA)/montmorillonite (CA-MMT), vermiculite nanoparticle/nanofiber, organo-vermiculites, Minocycline/Rifampin (M/R), Gentian Violet, Gardine, Gendine, Silver, Titanium-doped hydroxyapatite (Ti-Hap), Titanium-polybenzyl acrylate (PBA), polymer N,N-dimethyl-N-benzyl-N-(2-methacryloyloxyethyl) ammonium, acetate linked PLGA-glycol chitosan (GC) core-shell microspheres, Polyhexamethylenebiguanide hydrochloride (PHMB) polyactide (PLA) scaffolds, Zeolite (ZE) nanoparticles or glass ionomer, amorphous calcium phosphate (ACP) nanoparticles, gold nanoparticle/nanofiber, Vitamin E, and tetrapalmitate (TP). A coating of the solution can also be applied as a thin-layer film or series of layered films.

The solution comprising a biguanide derivative can also comprise a tissue adhesive or binding agent. Such adhesives can include biodegradable urethane isocyanoate derivatives such as TissuGlu (Cohera Medical, Inc.) and non-biodegradable glues such as cyanoacrylate glues (Dermabond).

The solution comprising a biguanide derivative can comprise from about 0.0001% to about 4% of the biguanide derivative by weight. For example, the solution can comprise from about 0.0001% to about 0.01%, from about 0.001% to about 0.1%, from about 0.01 to about 1%, from about 0.1 to about 4%, or from about 0.1% to about 0.15% biguanide derivative by weight. In specific examples, the solution can comprise about 0.0001%, 0.0002%, about 0.001%, about 0.005%, about 0.01%, about 0.02%, about 0.05%, about 0.1%, about 0.2%, about 0.5%, about 1%, about 1.5%, or about 2% biguanide derivative by weight.

The solution can have a pH of from about 5 to about 7.5, for example, about 5, 5.5, 6, 6.5, 7, or 7.5.

The solution comprising a biguanide derivative can, in some examples, further comprise an additional antimicrobial or antiseptic agent. Examples of suitable antimicrobials and antiseptic agents that can be used include hypochlorous acid, hypochlorous acid derivatives, chlorpactin (hypochlorous acid powder), triclosan, povidone, providone-iodine, PVP (polyvidone-iodine), polyhexanide (PHMB), octenidine dihydrochloride, magnolia bark derived extract: Magnolol, curcumin, N-Acetyl-L-Cysteine-Sodium Hydroxide (NALC-NaOH), and squalamine. The proportion of these additional agents can be about 0.001% to about 10%, about 0.01% to about 5%, about 0.1% to about 10%, or about 1% to about 5% by weight.

The solution comprising a biguanide derivative can, in some examples, further comprise one or more surfactants or foaming agents. As such, the solution comprising a biguanide derivative can be a chlorhexidine scrub or soap. The surfactants present may be anionic, nonionic, cationic and/or amphoteric or zwitterionic surfactants, the proportion of which in the compositions can be about 1% to about 70%, about 5% to about 50%, about 10% to about 30%, or about 1% to about 5% by weight. Typical examples of anionic surfactants are soaps, alkylbenzene sulfonates, alkanesulfonates, olefin sulfonates, alkyl ether sulfonates, glyceryl ether sulfonates, α-methyl ester sulfonates, sulfo-fatty acids, alkyl sulfates, fatty alcohol ether sulfates, glyceryl ether sulfates, fatty acid ether sulfates, hydroxy mixed ether sulfates, monoglyceride (ether) sulfates, fatty acid amide (ether) sulfates, mono- and alkoxylated and nonalkoxylated dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, for example acyl lactylates, acyl tartrates, acyl glutamates and acyl aspartates, alkyl oligoglucoside sulfates, alkyl oligoglucoside carboxylates, protein fatty acid condensates (especially wheat-based vegetable products) and alkyl (ether) phosphates. If the anionic surfactants contain polyglycol ether chains, these may have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolyzates (especially wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution.

Typical examples of cationic surfactants are quaternary ammonium compounds, for example dimethyldistearylammonium chloride or cetyltrimonium chloride, and ester quats, especially quaternized fatty acid trialkanolamine ester salts. Typical examples of amphoteric or zwitterionic surfactants are alkylbetaines, alkylamidobetaines, aminopropionates, aminoglycinates, imidazoliniumbetaines and sulfobetaines. The surfactants specified are exclusively known compounds. Typical examples of particularly suitable mild, i.e., particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkyl-amidobetaines, amphoacetals and/or protein fatty acid condensates, the latter preferably based on wheat proteins.

Examples of foaming agents include sodium laureth ether sulfate (SLES), sodium lauryl dodecyl sulfate (SDS), disodium laureth sulfosuccinate, ammonium lauryl sulfate (ALS), sodium pareth sulfate, and sodium coceth sulfate. Foaming agents can be present in the solution at from about 1% to about 70%, about 5% to about 50%, about 10% to about 30%, or about 1% to about 5% by weight.

The solution comprising a biguanide derivative can, in some examples, further comprise one or more antibiotics. Examples of antibiotics include amikacin, gentamicin, kanamycin, neomycin, streptomycin, tobramycin, bacitracin, clindamycin, daptomycin, lincomycin, linezolid, metronidazole, polymyxin, rifaximin, vancomycin, penicillin, cephalosporin, cephazolin, cephalexin, erythromycin, azithromycin, ciprofloxacin, levofloxacin, sulfadiazine, minocycline, tetracycline, and rifampin. Additional examples include “XF” (dicationic porphyrin structure) antibiotics. Additional examples include Teixobactin (cyclic undecapeptide), Teixobactin analogues, Oritavacin, Dalbavancin, Tedizolid, and antibacterial synthetic retinoids. The proportion of antibiotics can be about 0.001% to about 10%, about 0.01% to about 5%, about 0.1% to about 10%, or about 1% to about 5% by weight.

The solution comprising a biguanide derivative can, in some examples, further comprise additional agents such as acyclovir, cephradine, malphalen, procaine, ephedrine, adriamycin, dauno, mycin, plumbagin, atropine, quinine, digoxin, and quinidine, cephradine, cephalothin, cishydroxy-L-proline, melphalan, nicotinic acid, nitric oxide, nitroglycerin, chemodeoxycholic acid, chlorambucil, paclitaxel, sirolimus, 5-flurouracil, paclitaxel, mercaptoethanesulfonate, verapamil, or antifungal agents. The proportion of these additional agents can be about 0.001% to about 10%, about 0.01% to about 5%, about 0.1% to about 10%, or about 1% to about 5% by weight.

In other examples, the solution can further comprise angiogenesis inhibitors or growth factors. Examples of such suitable agents include epidermal growth factor, PDGF, VEGF, FGF, TNF, interleukins; interferons, anti-growth factors—antibodies, growth factor receptor-specific inhibitors, acitazanolast, iralukast, montelukast, pranlukast, verlukast, zafirlukast, and zileuton. The proportion of these additional agents can be about 0.001% to about 10%, about 0.01% to about 5%, about 0.1% to about 10%, or about 1% to about 5% by weight.

In some examples, the solution can further comprise anti-inflammatory agents. Examples of such agents include acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin, trolamine. Anti-inflammatory agents can be present in the solution at from about 1% to about 70%, about 5% to about 50%, about 10% to about 30%, or about 1% to about 5% by weight.

In a further example, the solution can further comprise titanium.

The solution comprising a biguanide derivative can be used to irrigate many different wounds or surgical sites. For example, irrigation with the solution comprising a biguanide derivative can be buccal (administration directed toward the cheek, generally from within the mouth); conjunctival (administration to the conjunctiva, the delicate membrane that lines the eyelids and covers the exposed surface of the eyeball); cutaneous (administration to the skin); endocervical (administration within the canal of the cervix uteri); endotracheal (administration directly into the trachea); infiltration (administration that results in substances passing into tissue spaces or into cells); interstitial (administration to or in the interstices of a tissue); intra-abdominal (administration within the abdomen); intra-articular (administration within a joint); intracartilaginous (administration within a cartilage); intracavernous (administration within a pathologic cavity); intracavitary (administration within a non-pathologic cavity); intraductal (administration within the duct of a gland); intralesional (administration within or introduced directly into a localized lesion); intraluminal (administration within the lumen of a tube); intramedullary (administration within the marrow cavity of a bone); intraocular (administration within the eye); intraperitoneal (administration within the peritoneal cavity); intrapulmonary (administration within the lungs or its bronchi); intra-nasal (administration within the nasal or periorbital sinuses); intrasynovial (administration within the synovial cavity of a joint); intratendinous (administration within a tendon); intrathoracic (administration within the thorax (internal to the ribs); synonymous with the term endothoracic); intratubular (administration within the tubules of an organ); intratumor (administration within a tumor); intrauterine (administration within the uterus); intravesical (administration within the bladder); intravitreal (administration within the vitreous body of the eye); irrigation (administration to bathe or flush open wounds or body cavities); laryngeal (administration directly upon the larynx); nasal (administration to the nose; administered by way of the nose); nasogastric (administration through the nose and into the stomach, usually by means of a tube); occlusive dressing (administration by the topical route which is then covered by a dressing which occludes the area); ophthalmic (administration to the external eye); oral (administration to or by way of the mouth); oropharyngeal (administration directly to the mouth and pharynx); percutaneous (administration through the skin); periarticular (administration around a joint); periodontal (administration around a tooth); rectal (administration to the rectum); respiratory (inhalation) (administration within the respiratory tract by inhaling orally or nasally for local or systemic effect); soft tissue (administration into any soft tissue); subconiunctival (administration beneath the conjunctiva); subcutaneous (administration beneath the skin; hypodermic; synonymous with the term subdermal); sublingual (administration beneath the tongue); submucosal (administration beneath the mucous membrane); topical (administration to a specific spot on the outer surface of the body. the term transmammary is a subset of the term); transmucosal (administration across the mucosa); ureteral (administration into the ureter); urethral (administration into the urethra); or vaginal (administration into the vagina).

In a specific example, irrigation with the solution comprising a biguanide derivative can be into the breast pocket or breast prosthesis capsule during breast surgery. The solution could also be applied by a spraying apparatus for dispersed coating of tissue or implant. Spraying can be by way of spray bottle, pump spray, pressurized spray, aerosol, nebulizer and the like.

Irrigating or coating the wound or surgical site can be accomplished by flushing the site with at least about 10 mL of the solution, for example at least about 50 mL, at least about 100 mL, at least about 150 mL, at least about 200 mL, at least about 250 mL, at least about 300 mL, at least about 500 mL, or at least about 1 L, or at least about 2 L. Irrigation or coating can be accomplished pouring or spraying the solution into the wound or surgical site. In the disclosed methods, the solution is not washed out of the wound or surgical site with water or saline. Excess solution can be suctioned out of the wound or surgical site. The wound or surgical site can then be closed, e.g., by sutures, staples, or glue, or covered with a bandage.

Because the solution comprising a biguanide derivative is not washed (flushed) out of the wound or surgical site, a significant portion remains in the wound or surgical site when closed. The amount of solution remaining can be at least 5 vol % of the amount used to irrigate the wound or surgical site, e.g., at least 10 vol %, 20 vol %, 30 vol %, 40 vol %, 50 vol %, 60 vol %, 70 vol %, 80 vol %, or 90 vol %.

It is also contemplated that the irrigation can uses two or more different biguanide solutions. For example, the use of an aqueous solution of a chlorhexidine salt with a strong negative charge can rapidly ionically bind to the positive charge of a silicone prosthesis. This salt solution can be made by the mixing of an aqueous solution of chlorhexidine gluconate and an inorganic phosphate salt such as sodium hexametaphosphate. Alternatively, other inorganic phosphate salts can be used and can include but are not limited to sodium metaphosphate and sodium tri-metaphosphate. The product formed is a chlorhexidine hexametaphosphate with a favorable cellular toxicity yet retaining a potent efficacy as an antiseptic, antimicrobial, anti-viral, anti-fungal, anti-fibroblast agent, anti-epithelial solution with both hydrophobic and hydrophilic, and ionically charged solution. Other aqueous forms of chlorhexidine-based solutions can be used with the combination of alternative/analog forms of chlorhexidine as well as other know antimicrobial or antiseptic agents. The solution can also be formulated in combination with a nanoparticle, nanofiber, glass-iomer, collagen, or linking agent for a predictable long-term elution. In a specific example, a first irrigation can use a solution comprising chlorhexidine gluconate and a second irrigation can use a solution comprising chlorhexidine hexametaphosphate.

The particular surgical site that can be irrigated or coated with the disclosed solutions can be a body cavity, breast pocket, orthopedic site (joint replacement), orthopedic site (bone fixation), or arthroscopy site, dental or craniofacial fixation site, or a traumatic wound.

Also disclosed herein are methods of cleaning or coating a surgical drain or surgical drain site comprising contacting the site with a solution comprising a biguanide derivative. The solution can be left in the surgical drain and/or surgical drain site for at least 10 minutes before rinsing or flushing with water. Alternatively, the solution can be left in place on the surgical drain and allowed to dry by evaporation.

### Articles

Also disclosed herein are articles, e.g., implants, coated with a biguanide derivative. The biguanide derivative can be any of those disclosed herein. By “coated” is meant a surface of the device or implant is covalently bound (directly or through a linking moiety) or ionically bound (through electrostatic interactions) to a biguanide derivative. “Coated” also means when a surface of the device or implant is coated with montmorillonite, nanoparticles, or nanofibers that have a biguanide derivative bound to, encapsulated within, or intercalated with the montmorillonite, nanoparticle, or nanofiber.

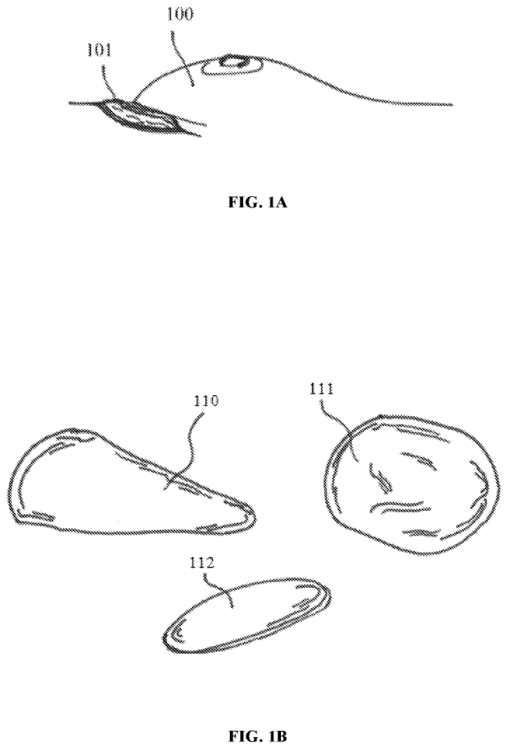

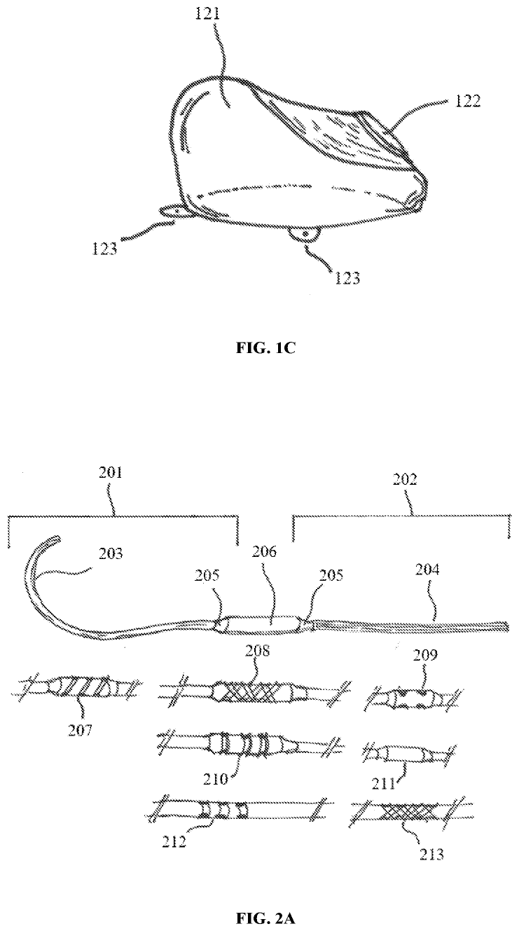

Coating the article with the disclosed compositions can reduce the presence of bacteria and risk of infection. The solution can also lubricate the article for ease of insertion/implantation of a prosthetic device (FIG. 1B and FIG. 1C), a breast implant (110, 111, and 112) or tissue expander (121) with a port site (122) or without a port site, and with anchoring tabs (123) or without anchoring tabs into a surgical cavity such a breast pocket (101, FIG. 1A).

Examples of devices that can be coated with a biguanide derivative can be drainage tubes (such as the ASPIRA Pleural Drainage Catheter from C.R. Bard), biliary T-tubes, clips, sutures, meshes, barriers (for the prevention of adhesions), anastomotic devices, conduits, packing agents, stents, staples, inferior vena cava filters, embolization agents, pumps (for the delivery of therapeutics), hemostatic implants (sponges), tissue fillers, cosmetic implants (breast implants, facial implants, prostheses), reconstructive implants (tissue expansion devices), bone grafts, skin grafts, intrauterine devices (IUD), ligatures, titanium implants (particularly in orthopedic joint replacement and bone fixation, dentistry, and oral-maxillofacial surgery), chest tubes, nasogastric tubes (such as the BARD Jejuna I Feeding/Gastric Decompression Tube from C.R. Bard), percutaneous feeding tubes (such as the BARD Button Replacement Gastrostomy Devices, the BARD PEG Feeding Devices, the DUAL PORT WIZARD Low-Profile Gastrostomy Device, FASTRAC Gastric Access Port, the GAUDERER GENIE System, the PONSKY Non-Balloon Replacement Gastrostomy Tubes, and the BARD Tri-Funnel Replacement Gastrostomy Tube from C.R. Bard), colostomy devices, bone wax, and Penrose drains, closed surgical drains (Jackson-Pratt drains), hair plugs, ear rings, nose rings, and other piercing-associated implants. In some examples the implant can be a prosthetic biologic mesh (acellular dermal matrix) or synthetic (polypropylene) mesh. Any foreign body when placed into the body is at risk for developing an infection-particularly in the period immediately following implantation and can thus be coated with the solutions disclosed herein.

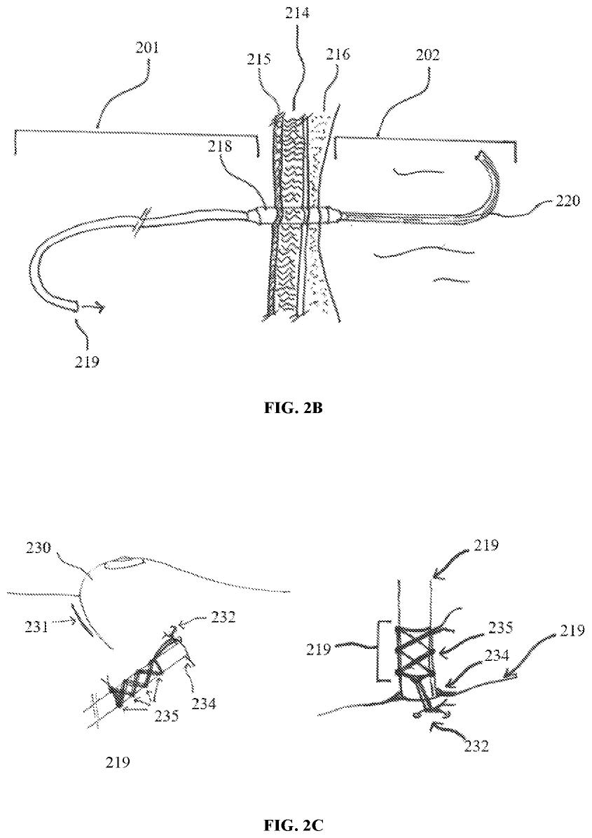

In specific examples, the article can be a surgical drain tube (FIG. 2A, FIG. 2B, and FIG. 2C). The drain can be coated with the biguanide derivative (e.g., a chlorhexidine-based coating), but can also be supplied without a coating and coated before use, or without a coating entirely. In further examples, the surgical drain can be manufactured with a length ranging from 5 cm to 35 cm, an outside diameter of 2-French to 18-French, and be fluted or non-fluted, and be attached to open or closed suction devices. The drain can possess a raised portion (206) 5 mm to 5 cm in length at the point of exit from the surgical cavity (234), muscle (216) subcutaneous tissue (214), and skin (215) with a larger diameter than the internal (202, 205, 204, 220) and external portions (201, 203, 205, 218, 219) of the drain designed to aid in the maintenance of a vacuum or viscous seal. The raised portion (206) can be 0.01 mm to 5 mm larger in diameter than the body of the drain. A portion of the drain at the point of dermal exit can contain a groove or channel or set of grooves or channels (207-213) either in a cylindrical or crisscrossing pattern, or an indention that is not fully circumferential or fully crisscrossed (207), to aid in securing the drain to the skin. In other embodiments the drain can possess crisscross (213) or cylindrical (212) grooves or channels without a raised portion of the drain. The anchoring stitch (232) or stiches can be tied around the drain with the loop or loops (235) of the suture material resting within the channel or set of channels (207-213). The grooves or channels aid in securing the suture material to the body of the drain (235) thereby preventing the drain from being dislodged or removed.

In other specific examples, disclosed herein is a cosmetic or reconstructive breast implant (FIG. 1B and FIG. 1C) coated with a biguanide derivative.

In further examples, disclosed herein is a nipple-areolar cover or dressing coated with a biguanide derivative. The biguanide derivative can be any of those disclosed herein, e.g., chlorhexidine hexametaphosphate, which binds to silicone and metallic materials. The breast implant can be a silicone breast implant, textured or untextured, shaped (110) or round (111), with or without gas pockets. The breast implant can also be a saline implant (112).

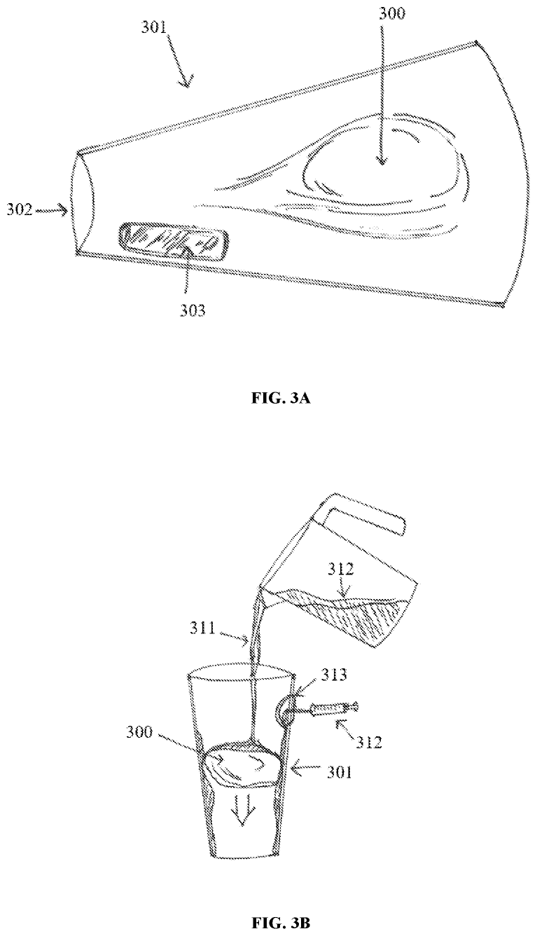

In further examples, the device can a “Keller Funnel” (FIG. 3A and FIG. 3B) or other funnel, cylindrical tube, or syringe like devices that injects silicone implants into the recipient cavity (101). The solution can be contained within a sealed sterile sleeve (303) within the device (e.g., InPlant Funnel) other form of sterile packaging. The solution contained within a sterile sealed sleeve (303) is designed to be manually or automatically broken to release the solution thereby coating the device (301) with an antimicrobial solution (312) prior to implant insertion through the funnel and into the surgical breast pocket (101). The antimicrobial solution may not be limited to a biguanide or biguanide derivative and may contain other antimicrobial agents such as povidone or povidone-iodine in either a solid, liquid, or gel form.

In further examples, the solution may be introduced into the delivery device by pouring (311) the solution directly into the device prior to use or introduced through a port within the device (313).

In further examples, the implant delivery device (FIG. 3A) is a conical, cylindrical, or tube-shaped device made of metal, nylon, mylar polyurethane, silicone, or other durable coating which lacks a lubricious coating whereby the solution when possessing a surfactant or sudsing agent is used as the lubricous coating agent on the funnel device surface.

In specific examples, the solution can also be used in conjunction with a precoated lubricious agent proprietary to a specific implant delivery device.

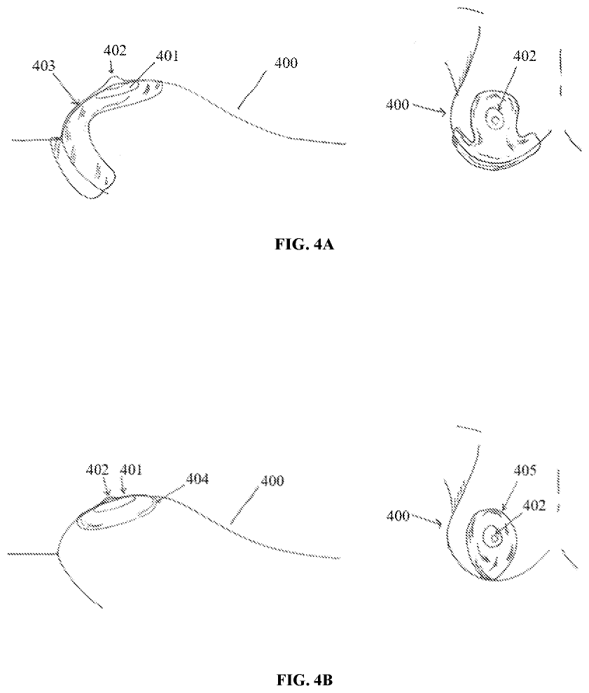

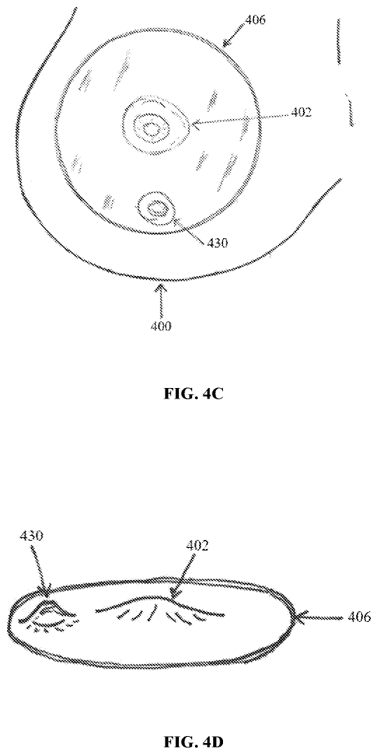

In further examples, disclosed herein is a nipple-areolar complex (NAC) cover or dressing (FIG. 4A, FIG. 4B, FIG. 4C, and FIG. 4D) coated with a biguanide derivative. The biguanide derivative can be any of those disclosed herein, e.g., chlorhexidine hexametaphosphate. For decolonization of the breast skin and NAC and to protect the breast surgical site and breast pocket during cosmetic and reconstructive breast surgery. The dressing can be molded with the raised impression (402) of a typical 5 cm NAC with the raised center (402) designed to fit snuggly over the NAC and overlying the immediate surrounding tissue. The dressing can be worn over the NAC for a fixed set of times between 24 hrs and 10 days to create a zone of antiseptic activity and promote local decolonization of the underlying breast ductal system within the breast parenchyma underlying the NAC. The dressing may also be used intraoperatively to protect the breast pocket from peri-operative contamination.

The dressing can be made of an occlusive and semi-occlusive material like a Tegaderm dressing. The dressing can be cut-out in an oval (405) or round shape (406) with 1-2 cm of material around the NAC in which a tissue adhesive can be applying to promote adherence to the skin surrounding the NAC. The middle of the dressing that would overlie the NAC can contain a chlorhexidine-based coating. A semi-liquid or gelatinous chlorhexidine-based coating could be utilized as well. The overall size of the dressing can be modified to include the typical incisions used during cosmetic breast surgery. Typical incision shapes include an ellipse (405) as in a circumvertical mastectomy or an anchor-shape used during a Wise-pattern incision.

In further examples, enclosed herein the aforementioned dressing can be a simple dressing in a circular, elliptical, square, or rectangular in shape coated with a biguanide derivative. The antimicrobial solution may not be limited to a biguanide or biguanide derivative and may contain other antimicrobial agents such as povidone or povidone-iodine in either a solid, liquid, or gel form. The occlusive dressing can be applied to a surgical or traumatic wound, not limited to the NAC, to promote antimicrobial activity during wound healing.

Additionally, to ease the removal of the dressing, a raised blister or a molded tab (430) can be incorporated. The blister or tab can be placed towards the outer portion of the dressing. The wearer of health-care professional would simply lift on the tab or blister (430) to pull up on the dressing thereby easing its removal.

The optimal coating of a medical implant or medical implant delivery system can employ a compound with an initial burst pattern of elution for antibacterial action at time of surgical exposure as well as long-term predictable release that would act to prevent biofilm formation from endogenous source such as breast tissue during mammoplasty augmentation and reconstruction. Additionally, the desirable substance when eluded would have a cytotoxicity profile against fibroblast which have been implicated in the formation of capsular contracture.

Also disclosed are kits comprising a medical device, such as a drain, port, suture, dressing, funnel, implant and a pouch or bottle comprising solution comprising a biguanide derivative as disclosed herein.

Coating the article can be accomplished by simply spraying or contacting the article with the solution, or by submerging the article in the solution comprising a biguanide derivative. Alternatively, the solution can be formulated to contain an evaporative substance such as alcohol. A foaming agent can be included as well. The solution can also include a combination of sodium hexametaphosphate, or other buffering inorganic salt, at a concentration of 0.01% to 99.9% in chlorhexidine gluconate. This can yield a solution chlorhexidine gluconate containing a partial positive charge to bind the lipopolysaccharide groups on bacterial cell walls, and would also allow for negatively charged chlorhexidine hexametaphosphate to bind to Silicone tubing. Chlorhexidine metaphosphate would also readily bind titanium comprising orthopedic joint replacement and maxillomandibular fixation hardware. Other pharmacological agents and chlorhexidine derivatives can be used in addition to chlorhexidine gluconate to optimize efficacy and silicone binding, as well as titanium binding.

1-30. (canceled)

31. A surgical device comprising:
a delivery device configured for inserting an implant into a surgical site; and
a sealed container prepositioned internal to the delivery device, wherein the sealed container comprises an antimicrobial solution.

32. The surgical device of claim 31, wherein the sealed container is a sterile sealed sleeve.

33. The surgical device of claim 31, wherein the antimicrobial solution further comprises a surfactant.

34. The surgical device of claim 33, wherein the surfactant comprises an alkylbenzene-sulfonate.

35. The surgical device of claim 31, wherein the antimicrobial solution further comprises a foaming agent.

36. The surgical device of claim 35, wherein the foaming agent comprises at least one of sodium laureth ether sulfate, sodium lauryl dodecyl sulfate, disodium laureth sulfosuccinate, ammonium lauryl sulfate, sodium pareth sulfate, and sodium coceth sulfate.

37. The surgical device of claim 31, wherein the antimicrobial solution further comprises an antibiotic.

38. The surgical device of claim 37, wherein the antibiotic includes at least of gentamicin, bacitracin, and cefazolin.

39. The surgical device of claim 31, wherein the antimicrobial solution further comprises at least one of hypochlorous acid and polyvidone-iodine.

40. The surgical device of claim 31, wherein the antimicrobial solution further comprises at least one of chlorhexidine, and hexametaphosphate.

41. The surgical device of claim 31, wherein the sealed container is configured to be manually broken to release the antimicrobial solution prior to inserting the implant into the delivery device.

42. The surgical device of claim 31, wherein the sealed container is configured to be automatically broken to release the antimicrobial solution prior to inserting the implant into the delivery device.

43. The surgical device of claim 31, wherein the antimicrobial solution comprises a biguanide derivative.

44. The surgical device of claim 31, wherein the antimicrobial solution comprises chlorhexidine.

45. The surgical device of claim 31, wherein the antimicrobial solution comprises chlorhexidine gluconate soap.

46. The surgical device of claim 31, wherein the delivery device is a funnel.

47. The surgical device of claim 46, wherein the delivery device is at least one of a Keller funnel and an Inplant funnel.

48. The surgical device of claim 31, wherein the delivery device is a cylindrical tube.

49. The surgical device of claim 31, wherein surgical site is a surgical breast pocket.

50. The surgical device of claim 31, wherein surgical site is a body cavity.

51. The surgical device of claim 31, wherein surgical site is a traumatic wound.

52. The surgical device of claim 31, wherein surgical site is at least one of an orthopedic site and an arthroscopy site.

53. The surgical device of claim 31, wherein the implant is a silicone implant.

54. The surgical device of claim 31, wherein the implant is a saline implant.

55. The surgical device of claim 31, wherein the implant is a polyurethane implant.

56. The surgical device of claim 31, wherein the implant is a silicone tissue expander.

57. The surgical device of claim 31, wherein the implant is a polyurethane tissue expander.

58. The surgical device of claim 31, wherein the implant is at least one of a prosthetic biologic mesh and a synthetic mesh.

59. A method of operating a surgical device, the method comprising:
inserting the surgical device into a surgical site, wherein the surgical device comprises:
a delivery device configured for inserting an implant into the surgical site; and
a sealed container prepositioned internal to the delivery device, wherein the sealed container comprises an antimicrobial solution.
and
automatically releasing the antimicrobial solution to pre-coat an internal surface of the delivery device prior to inserting the implant; and
manually inserting the implant into the delivery device.

60. A method of operating a surgical device, the method comprising:
inserting the surgical device into a surgical site, wherein the surgical device comprises:
a delivery device configured for inserting an implant into the surgical site; and
a sealed container prepositioned internal to the delivery device, wherein the sealed container comprises an antimicrobial solution.
and
manually releasing the antimicrobial solution to pre-coat an internal surface of the delivery device prior to inserting the implant; and
manually inserting the implant into the delivery device.
